(19) United States
(12) Patent Application Publication
(10) Pub. No.: US 2005/0137652 A1
(43) Pub. Date: Jun. 23, 2005

(54) System and method for interfacing cellular matter with a machine

(75) Inventors: Lawrence James Cauller, Plano, TX (US); Erik Nilsen, McKinney, TX (US)

Correspondence Address: HAYNES AND BOONE, LLP, 901 MAIN STREET, SUITE 3100, DALLAS, TX, 75202, US

(73) Assignees: The Board of Regents of the University of Texas at Dallas, Richardson, TX (US); Zyvex Corporation, Richardson, TX (US)

(21) Appl. No.: 10/741,136

(22) Filed: Dec. 19, 2003

(51) Int. Cl. A61N 1/08

(52) U.S. Cl. 607/60

(57) ABSTRACT

A system for interfacing cellular matter with a machine comprising a transponder implantable in cellular matter and configured to detect a first signal. The system also includes a receiver external to the cellular matter and configured to receive a second signal in response to the transponder detection of the first signal. A transmitter is also included in the system to transmit a third signal to a machine in response to the receiver receipt of the second signal.

### BACKGROUND

The present disclosure relates generally to the field of interfacing cellular matter with a machine and, more specifically, to a system and method for cellular matter-machine interfacing involving the detection of signals in cellular matter.

Cellular matter-machine interfacing offers possible solutions to a wide variety of problems. For example, thousands of people suffer from a variety of disorders that disconnect the brain from its inputs or outputs, including amyotropic lateral sclerosis, paralysis due to spinal-cord injury, cerebral palsy, polio, or sensory loss such as blindness or deafness. By allowing for machine control through the use of brain signals, cellular matter-machine interfacing systems offer these people the hope of walking again and/or actively engaging with the world. In addition to gaining or restoring lost functionality, cellular matter-machine interfacing systems may be used to enhance human functionality and performance in a wide variety of applications, such as enabling pilots to perform complex aerial maneuvers with greater efficiency.

Several issues arise when implementing cellular matter-machine interfacing systems, including in interfacing applications involving the human brain. Systems interfacing with brain tissue are highly invasive, usually requiring electrodes to be implanted into the brain. These relatively large electrodes are typically connected to external devices by transcranial wires that pass through the protective coverings of the brain and skull. The size of the electrodes and the attendant transcranial wiring both increase the risk of brain-tissue damage, thereby increasing the risk of brain infection, permanent brain damage, and other life-threatening medical problems.

Furthermore, powering the electrodes may require battery implantation in the brain tissue and/or additional wiring, increasing the risk of brain-tissue damage to an even greater degree. Other issues include surgical complexity, the unwanted movement of interfacing-system devices within the brain, and the sensitivity of the interfacing system to head or body movement. Although cast in terms of brain-control applications, one or more of these problems are present in most cellular matter-machine interfacing systems, regardless of where the cellular matter is located.

Accordingly, what is needed in the art is a cellular matter-machine interface system, device and/or method that addresses the above-discussed issues.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 illustrates a block diagram of one embodiment of a cellular matter-machine interface system constructed according to aspects of the present disclosure.

FIG. 2 illustrates a block diagram of another embodiment of a cellular matter-machine interface system constructed according to aspects of the present disclosure.

FIG. 3 illustrates a front view of one embodiment of a helmet device constructed according to aspects of the present disclosure.

FIG. 4 illustrates a detailed view of a portion of the helmet shown in FIG. 3.

FIG. 5 illustrates a perspective view of an embodiment of a hand-held scanning device constructed according to aspects of the present disclosure.

FIG. 6 illustrates a perspective view of an embodiment of a table-top scanning device constructed according to aspects of the present disclosure.

FIG. 7 illustrates a functional schematic of one embodiment of a transponder constructed according to aspects of the present disclosure.

FIG. 8 illustrates one embodiment of a circuit diagram of the transponder shown in FIG. 4.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

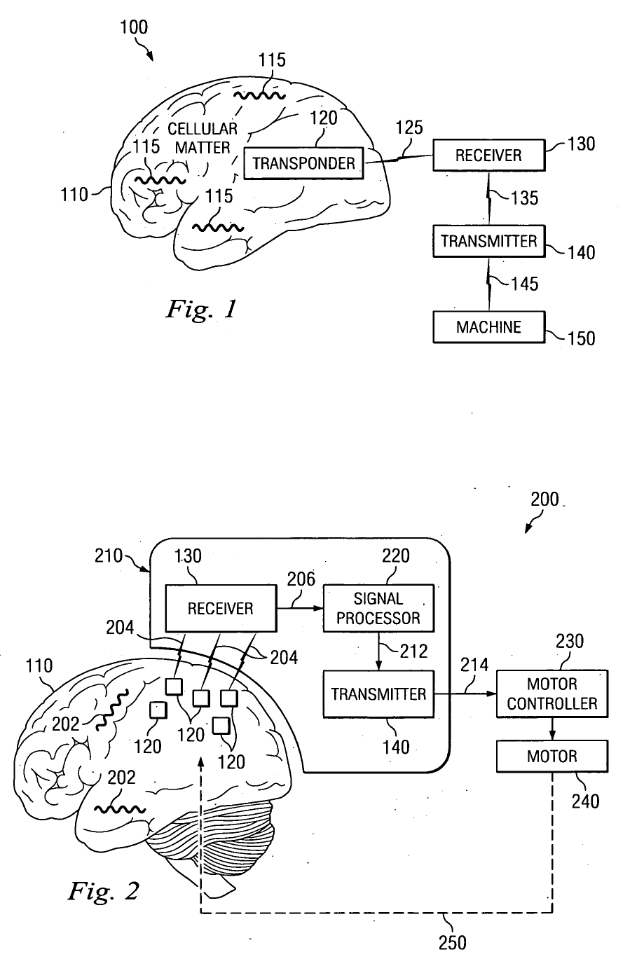

Referring to FIG. 1, illustrated is a block diagram of one embodiment of a cellular matter-machine interface system 100 constructed according to aspects of the present disclosure. The cellular matter-machine interface system 100 includes a transponder 120 implanted in or proximate cellular matter 110 in an orientation encouraging the detection of a signal 115 propagating in the cellular matter 110. The signal 115 may be a biological signal. For example, the signal 115 may be a trans-membrane current, such as that generated by neuronal unit activity. In one embodiment, the signal 115 is generated by charged ions propagating between cells in the cellular matter 110.

The transponder 120 transmits a signal 125 to a receiver 130 in response to the detection of the signal 115. The receiver 130 transmits a signal 135 to a transmitter 140 in response to receiving the signal 125. The transmitter 140 then sends a signal 145 to a machine 150. Thus, the transmitter 140 may send the signal 145 to the machine 150 in response to the detection of the signal 115 by the transponder 120. The detection of the signal 115 by the transponder 120 may include detecting the existence of the signal 115 or, in one embodiment, detecting that a voltage or other electrical characteristic of the signal 115 reaches or exceeds a threshold value. For example, the transponder 120 may detect when the signal 115 reaches or exceeds about 0.1 mV.

Referring to FIG. 2, illustrated is a block diagram of another embodiment of the cellular matter-machine interface system 100 constructed according to aspects of the present disclosure, herein designated by the reference numeral 200. In the illustrated embodiment, the cellular matter 110 is depicted as human brain tissue, although aspects of the present disclosure are applicable and/or readily adaptable to other types of cellular matter, including muscle, blood, bone, fat, lymph nodes, nerves, water, partially digested food, cartilage, skin, hair roots, synovial fluids, mucous, pericardial fluids, spinal fluid, etc.

A plurality of transponders 120 implanted in the cellular matter 110 are configured to detect signals in the cellular matter 110 (“cellular matter signals 202”). As each of the transponders 120 detects a cellular matter signal 202, the transponders 120 generate and transmit another signal (“transponder signals 204”) to the receiver 130. Each of the transponder signals 204 may be a function of a cellular matter signal 202, may include a cellular matter signal 202, or may be or replicate the cellular matter signal 202. In one embodiment, one or more of the transponder signals 204 may comprise a cellular matter signal concatenated or multiplexed with additional information, such as identification of the particular transponder 120 that detected the cellular matter signal 202 or generated the transponder signal 204.

As in the system 100 discussed above, the receiver 130 is configured to transmit signals (“receiver signals 206”) to the transmitter 140 upon receipt of the transponder signals 204. In the illustrated embodiment, the receiver 130 and the transmitter 140 are housed in a helmet device 210 worn on the head of a human. However, in other embodiments, the receiver 130 and the transmitter 140 may be co-located in other components, such as in a wand or other hand-held device, as in embodiments discussed below. The system 200 may also include a signal processor 220 co-located with and electrically interposing the receiver 130 and the transmitter 140. The signal processor 220 processes the receiver signal 206 before transmitting a processed signal 212 to the transmitter 140, such as by amplification, noise reduction, etc.

The transmitter 140 sends signals (“transmitter signals 214”), which may be, include, or be a function of the receiver signals 206 or the processed signals 212, to a motor controller 230 configured to control a motor 240. The motor 240 may be configured to impart motion to and/or direct motion of a wheelchair, an automobile, an aircraft, a watercraft, or other mechanized apparatus. Further examples include the transmitter 140 sending signals that are used to control robotic actuators, navigational systems, graphical display units, music synthesizers and speech synthesizers. The transmitter 140 may also generate and/or transmit signals employed to instruct a computer to execute a variety of commands. Although not limited by the scope of the present disclosure, such a computer may be a personal computer, a personal digital assistant, a home automation system controller, a telephone and/or other electronic devices.

In one embodiment, the system 200 may include a feedback loop 250. For example, in an embodiment in which the motor 240 controls the motion of a wheelchair, the feedback loop 250 may include sensors that detect the human sensing of movement of the wheelchair caused by the motor 240. Thus, the feedback loop 250 may include additional transponders implanted in the cellular matter 110 and similar to the transponders 120 to detect additional signals in the cellular matter 110 generated in response to the motion of the wheelchair. Furthermore, the sensing by the human or other organism may take place in a variety of modes or combination of modes, including sight, sound, touch or a combination thereof. Factors in addition to or as an alternative to the result of the operation of the machine 150 may be employed to generate signals in the cellular matter 110 as input for the feedback loop 250, such as other operations by other machines, organism-to-organism interactions and environmental factors.

As discussed above, it is understood that the cellular matter 110 may be in a human part other than the brain, and that it may also be in non-human organisms, such as lab-animals, animal assistants (e.g., seeing-eye dogs), wildlife (e.g., for tracking and/or disease/health control), artificial organs and other organisms. Furthermore, it is understood that a hand-held device such as a wand may be used to house the receiver 130, the transmitter 140, and/or the signal processor 220. The support structure of a diagnostic machine, such as an X-ray machine or a computed tomography (CT) scan machine, may also house the receiver 130, the transmitter 140, and/or the signal processor 220, as in embodiments discussed below. A polygraph or other device employed in lie detection is another environment in which aspects of the present disclosure may be implemented.

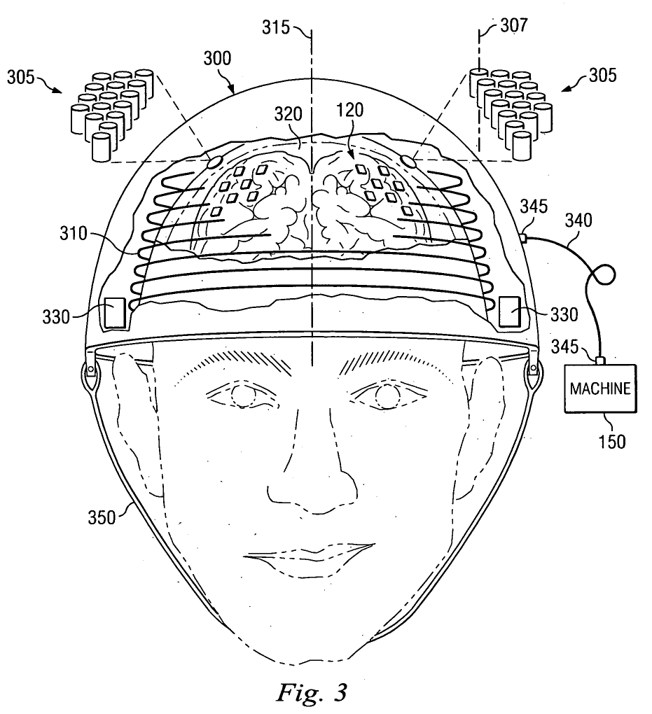

Referring to FIG. 3, illustrated is a front view of one embodiment of the helmet device 210 shown in FIG. 2, herein designated by reference numeral 300. The helmet device 300 includes one or more arrays of receivers 305 which, as in the illustrated embodiment, may be or comprise reader coils positioned in various areas around a human head 320. The receiver 130 shown in FIGS. 1 and/or 2 may be embodied in the helmet device 300 as one or more of the receivers 305. A power coil 310 is also included in the helmet device 300, substantially encircling the human head 320 about an axis 315. The axis 315 may be substantially parallel with the brain stem in the human head 320, substantially perpendicular to the brain stem, or oriented at an angle relative to the brain stem ranging between about 0° and about 90°. In one embodiment, the longitudinal axes 307 of one or more of the receiver reader coils 305 may also be substantially parallel to the axis 315 of the power coil 310.

The helmet device 300 may also include an embodiment of the transmitter 140 shown in FIGS. 1 and 2, herein designated by the reference numeral 330. The transmitter 330 is in electrical communication with one or more of the receivers 305, such as by hard-wiring or, as shown in FIG. 3, by RF or other wireless communication. The helmet device 300 may also include more than one transmitter 330. The helmet device 300 is in electrical communication with a machine 150 (schematically shown in FIG. 3) or a motor controller thereof, as discussed above with reference to FIGS. 1 and 2. Such communication may be wireless or, as shown, by a cord or other electrical cable 340. The electrical cable 340 may include detachable electrical connectors 345, such that the helmet device 300 may remain affixed to the human head 320 despite lack of proximity to the machine 150. The helmet device 300 may also include a chin strap 350 or other means for maintaining the receivers 305 in a fixed orientation relative to transponders 120 implanted in the human head 320.

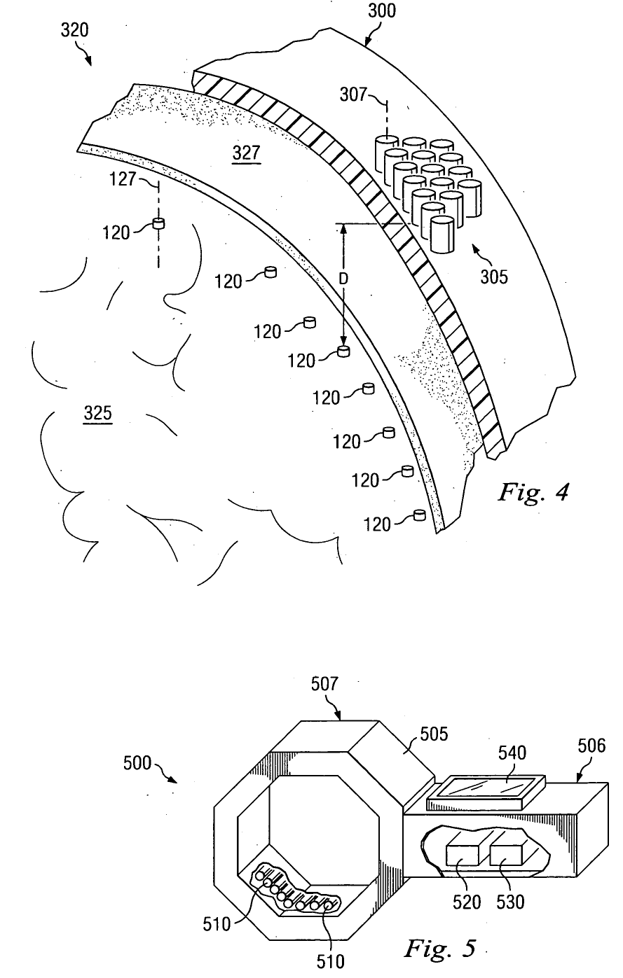

Referring to FIG. 4, illustrated is a detailed view of a portion of the helmet device 300 shown in FIG. 3 arranged on the human head 320. The human head 320 includes brain tissue 325 confined within a boundary 327 comprising the skull and scalp. An array of transponders 120 is wholly implanted into the brain tissue 325 beneath the boundary 327. An array of the reader coils and/or other type of receivers 305 within the helmet device 300 are located adjacent or proximate a surface of the boundary 327. In one embodiment, such as in an embodiment discussed below in which the transponder 120 comprises a transponder coil, each of the transponders 120 and the receivers 305 are oriented such that their respective axes 127, 307 are substantially parallel. The offset spacing D between each of the transponders 120 and a closest one of the receivers 305 may be about 3 centimeters, although other offset spacings may also be employed within the scope of the present disclosure. It is understood that other offset spacings may be necessary depending upon the physical and/or electrical properties of the receivers 305 and the transponders 120.

Each of the transponders 120 may be magnetically coupled with at least one of the receivers 305. Accordingly, each of the transponders 120 may send an signal to at least one of the receivers 305, possibly employing radio-frequency propagation via the magnetic coupling. Each of the transponders 120 may also be magnetically coupled with the power coil 310 (shown in FIG. 3). The power coil 310 may power the transponders 120 using radio-frequency propagation via the magnetic coupling. Thus, each of the transponders 120 may send an signal to at least one of the receivers 305 without interrupting the magnetic coupling of the power coil 310 and the transponders 120.

It is understood that a transponder 120 may send a signal to at least one of the receivers 305 using other means, such as electric-field coupling. Further, it is understood that the power coil 310 may power the transponders 120 using other means, such as electric-field coupling. Such a configuration may allow the power coil 310 to be removed from the helmet device 300 and be located elsewhere. Moreover, the reader coils 305 may be employed to receive signals from the transponders 120 and to power the transponders 120, a configuration that may make the power coil 310 unnecessary.

Referring to FIG. 5, illustrated is a perspective view of one embodiment of a hand-held scanning device 500 constructed according to aspects of the present disclosure. The hand-held scanning device 500 is an alternative environment in which aspects of the helmet device 300 shown in FIGS. 3 and 4 may be implemented. For example, the hand-held scanning device 500 includes one or more receivers 510 which may comprise reader coils or otherwise be substantially similar to the receivers 305 shown in FIGS. 3 and 4, and also includes a transmitter 520 which is substantially similar to the transmitter 330 shown in FIG. 3. The receivers 510 and the transmitter 520 are enclosed in a housing 505 having a handle portion 506 and a scanning portion 507. The handle portion 506 is configured to be grasped by a single human hand and oriented proximate a human limb or other region having implanted transponders according to aspects of the present disclosure. The scanning portion 507 is configured to allow the human limb having the implanted transponders to pass therethrough. Thus, the scanning portion 507 has an annulus shape containing the receivers 510. In another embodiment, the scanning portion 507 may be substantially planar or U-shaped. In operation, the hand-held scanning device 500 is passed over the region of cellular matter implanted with transponders.

The transmitter 520 is in wired or wireless electrical communication with the receivers 510. The hand-held device 500 may also include a processor 530 configured to analyze the signals received by the transmitter 520 (i.e., the processor 530 may be the machine 150 in the interface system 100 shown in FIG. 1). The processor 530 may be configured to transmit analysis results and display data to a display screen 540 remote from the hand-held device 500 or, as in the illustrated embodiment, integral to the hand-held device 500.

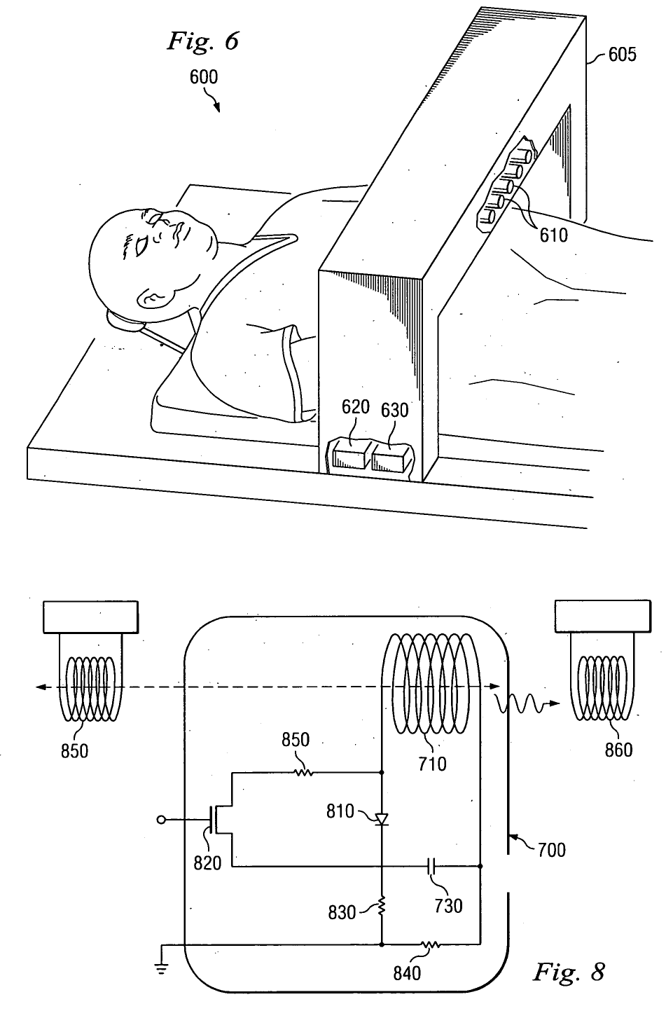

Referring to FIG. 6, illustrated is a perspective view of an embodiment of a stationary scanning device 600 constructed according to aspects of the present disclosure. The stationary scanning device 600 is an alternative environment in which aspects of the helmet device 300 shown in FIGS. 3 and 4 may be implemented. For example, the stationary scanning device 600 includes one or more receivers 610 which may comprise reader coils or otherwise be substantially similar to the receivers 305 shown in FIGS. 3 and 4, and also includes a transmitter 620 which is substantially similar to the transmitter 330 shown in FIG. 3. The receivers 610 and the transmitter 620 are enclosed in a housing 605 configured to allow a substantial portion of a human body to pass therethrough. In operation, the housing 605 is passed over a region of cellular matter implanted with transponders, or the implanted region is passed under the housing 605.

The transmitter 620 is in wired or wireless electrical communication with the receivers 610. The stationary device 600 may also include a processor 630 configured to analyze the signals received by the transmitter 620 (i.e., the processor 630 may be the machine 150 in the interface system 100 shown in FIG. 1). The processor 630 may be configured to transmit analysis results and display data to a display screen remote from or integral to the stationary device 600.

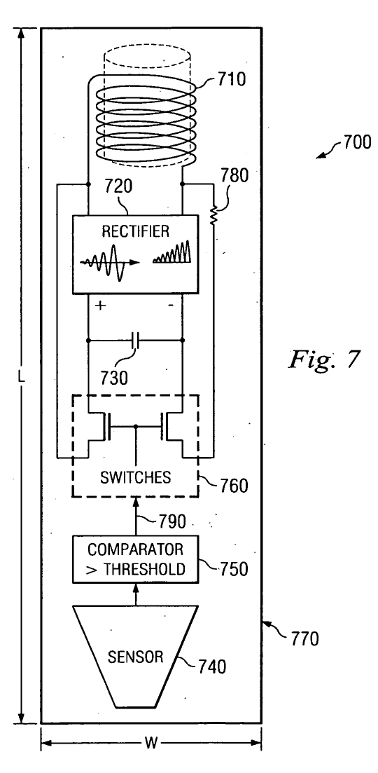

Referring to FIG. 7, illustrated is a functional schematic of one embodiment of a transponder 700 constructed according to aspects of the present disclosure. The transponder 700 may be employed within cellular matter-machine interface systems constructed according to aspects of the present disclosure. That is, the transponder 700 may be substantially similar to the transponders 120 discussed above with reference to FIGS. 1-4. Moreover, those skilled in the art will readily recognize that the embodiment shown in FIG. 7 is exemplary, and that myriad alternative circuits and/or systems may be employed as a transponder within a cellular matter-machine interface system within the scope of the present disclosure.

The transponder 700 includes a coil 710 oriented vertically and located towards the top of the transponder 700 (as viewed in FIG. 7). Magnetic coupling between a power coil and the transponder 700, as in embodiments described above, may be accomplished with the transponder coil 710. Likewise, the magnetic coupling between at least one receiver reader coil and the transponder 700, as in embodiments described above, may be accomplished with the transponder coil 710.

The transponder coil 710 is coupled in parallel to a rectifier 720 and a capacitor 730. The transponder 700 also includes a sensor 740, a comparator 750, and a pair of switches 760. A resistor 780 is in electrical communication with the pair of switches 760. The sensor 740 may be located opposite or distal from the coil 710. The transponder 700 is enclosed in a housing 770 which, in one embodiment, is substantially coated with a bioprotectant that is compatible with the cellular tissue in which implanting is contemplated (e.g., human brain tissue). In one embodiment, the length L of the housing 770 may be about 1000 microns and the width (or diameter) W may be about 600 microns. In another embodiment, the length L of the housing 770 may be about 200 microns and the width (or diameter) W may be about 50 microns. The housing 770 may have a maximum lateral dimension (i.e., L or W) of about 200 microns. In another embodiment, the maximum lateral dimension may be about 1000 microns.

The transponder 700 may have three modes: a power mode, a transmission mode, and an idle mode. In regard to the power mode, the transponder 700 may be powered by radio frequency propagation via the magnetic coupling of the transponder coil 710 to an external coil such as the power coil 310 or the receiver reader coil 305 of FIG. 3, inducing a voltage across the transponder coil 710. This voltage may be rectified by the rectifier 720 and subsequently employed to store a charge in the capacitor 730. In one embodiment, the charging time of the capacitor may be about 10 milliseconds.

In regard to the transmission mode of the transponder 700, the sensor 740 may detect a signal in cellular matter. The signal may be a biological signal. The comparator 750 may compare this signal to a threshold signal level. If the signal detected in cellular matter is greater than the threshold signal, the transponder may enter transmission mode. A trigger signal 790 may be sent to the switches 760, which in turn are switched “on,” causing the capacitor 730 to discharge and current to flow through the transponder coil 710 and the resistor 780. The current through the transponder coil 710 may cause a signal to be transmitted to an external receiver such as the receiver 305 via magnetic coupling. In one embodiment, the discharge of the capacitor may be rapid, with a discharge time of about 50 nanoseconds (a “burst transmission” mode). It is understood that other transmission modes with differing discharge times may be employed.

In regard to the idle mode of the transponder 700, the transponder 700 may sit idle when the capacitor 730 is fully charged and the switches 760 are switched “off.” During the idle mode, the sensor 740 may be continually detecting signals in cellular matter and the comparator 750 may be continually comparing these signals to the threshold signal level. As long as the comparator 750 determines that each cellular-matter signal detected by the sensor 740 is not greater than the threshold signal level, no trigger signal 790 is sent to the switches 760 and the switches 760 remain “off.”

It is understood that two capacitors may be employed in the transponder 700, one capacitor employed in powering the transponder 700 and one capacitor employed in transmitting a signal from the transponder 700. It is also understood that signals other than the trigger signal 790 may be sent from the comparator 750 to the switches 760.

Referring to FIG. 8, with continued reference to FIG. 7, illustrated is a schematic view of another embodiment of the transponder 700 shown in FIG. 7. In the embodiment shown in FIG. 8, the transponder 700 includes a diode 810 substituted for the rectifier 720 of FIG. 7. The transponder coil 710 may be in electrical communication with the diode 810, which in turn may be in electrical communication with the capacitor 730.

A metal-oxide semiconductor field-effect transistor (MOSFET) or other type of transistor 820 may be in electrical communication with the capacitor 730, the transponder coil 710, and a resistor 850. Additionally, a resistor 830 and a resistor 840 may also be in electrical communication with the transistor 820 to assist with biasing the transistor 820. Due to these electrical communications, the transistor 820 may be substituted for the sensor 740, the comparator 750, and the switches 760 shown in FIG. 7, and the resistor 850 may be substituted for the resistor 780 shown in FIG. 7.

The magnetic coupling of the transponder coil 710 and a power coil 850 (which may be substantially similar to the power coil 310 shown in FIG. 3), and of the transponder coil 710 and at least one of a plurality of receiver reader coils 860 (which may be substantially similar to the receiver reader coils 305 shown in FIG. 3), is also depicted in FIG. 8.

It is understood that the symbols in FIGS. 7 and 8 representing various circuit components and the physical relationships among the components are not intended to indicate the actual physical locations of the components. It is also understood that a transponder constructed according to aspects of the present disclosure may include additional components, such as a micro-antenna for electric-field coupling to a signal detector external to cellular matter.

In one embodiment, the transistor 820 may have a negative threshold potential and be insulated by a thin film of silica. The transistor 820 may be a p-channel junction field-effect transistor (p-channel JFET) having a source, a gate, and a drain, and may be biased at 1 volt drain-source (V_{DS}). The negative fields generated in cellular matter may be on the order of 0.1 to 1 millivolts (mV). Such negative fields may gate a drain-source current (I_{DS}) for up to 100 nanoamps (nA). It is understood that an external surface of the gate of the transistor 820 or an external surface of the transponder 700 may be post-processed with a material that promotes cellular growth, ensuring ohmic contact between cellular matter and the external surface.

The transponder 700 may be powered by radio-frequency propagation via the magnetic coupling of the power coil 850 and the transponder coil 710, as discussed above. This magnetic coupling may induce a voltage across the transponder coil 710. This powering process may include current flowing from the transponder coil 710 to the capacitor 730 through the diode 810, charging the capacitor 730. The charging time of the capacitor 730 may be about 10 milliseconds.

The cellular matter may generate negative fields detectable by the transistor 820 at its gate when the transistor 820 is employed as the sensor 740. If the absolute value of the negative field potential at the gate of the transistor 820 is greater than the absolute value of the negative threshold potential of the transistor 820, the capacitor 730 may be discharged and a drain-source current (I_{DS}) may flow from the drain of the transistor 820 for a relatively small period of time. In this manner the transistor 820 is first acting as the sensor 740, then as the comparator 750, and then as the set of switches 760, the switches being “on.” The drain-source current may flow through the transponder coil 710, causing a signal to be transmitted to at least one receiver reader coil 860 by radio-frequency propagation via the magnetic coupling of the transponder coil 710 and the receiver reader coil 860.

When the capacitor 730 is fully charged and there is no drain-source current flowing from the transistor 820, the transponder 700 may sit idle. The signal transmitted from the transponder coil 710 to the receiver reader coil 860 may be filtered, amplified and/or demodulated to reconstruct desired information, such as when the negative field was generated temporally, the implanted location of the transponder 700 that sent the signal, etc.

In one embodiment, the power coil 850 may be omitted or dormant and the receiver reader coil 860 may be used to remotely power the transponder 700 and to receive signals from the transponder 700. The reader coil 860 may have a three-centimeter radius and utilize three turns of number 40 gage copper wire, resulting in an inductance of about 1.2 microhenries, a resistance of about 2 ohms, and a self-resonant frequency that is greater than 250 MHz for operation at 100 MHz. At a radio-frequency power dissipation level of 120 milliwatts, the current flowing through the receiver reader coil 860 may be 250 milliamps.

The transponder coil 710 may have a 300-micron radius and utilize 20 turns of 40-micron gold wire, resulting in an inductance of about 116 nanohenries, a direct-current resistance of about 0.76 ohms, and a self-resonant frequency of about 8 GHz. Due to the physical properties of the gold wire, the radio-frequency resistance of the transponder coil 710 may be about 0.87 ohms. The capacitance of the transponder 700 may be about 22 picofarads and the load resistance of the transponder 700 may be about 50 kilo-ohms. The transponder 700 may have an overall volume that is less than or about 1 cubic millimeter and a maximum lateral dimension of less than about 1000 microns.

Thus, the present disclosure introduces a system for interfacing cellular matter with a machine comprising, in one embodiment, a transponder implantable in cellular matter and configured to detect a first signal. The system also includes a receiver external to the cellular matter and configured to receive a second signal in response to the transponder detection of the first signal. A transmitter is also included in the system to transmit a third signal to a machine in response to the receiver receipt of the second signal. Another embodiment of a system for interfacing cellular matter with a machine introduced herein comprises means implantable in cellular matter for detecting a first signal in the cellular matter, means for receiving a second signal transmitted in response to the detection of the first signal, and means for transmitting a third signal to a machine in response to the receipt of the second signal.

A transponder is also provided in the present disclosure, the transponder being implantable in cellular matter and comprising a remote-activated power source and a sensor powered by the power source and configured to detect a first signal propagating in cellular matter. The transponder also includes a transmitter configured to transmit a second signal in response to the sensor's detection of the first signal.

A method for controlling a machine in response to signals propagating in cellular matter is also introduced in the present disclosure, the method comprising detecting a first signal in cellular matter and transmitting a second signal in response to the detection of the first signal. The method also includes transmitting a third signal to a machine in response to the receipt of the second signal.

An interface between cellular matter and a machine is also introduced in the present disclosure, the interface comprising a housing and a receiver coupled to the housing, the receiver being configured to receive a first signal from a transponder implanted in cellular matter, wherein the receiver is external to the cellular matter. The interface also includes a transmitter coupled to the housing and configured to transmit a second signal to a machine in response to the receiver receipt of the first signal.

The foregoing has outlined features of several embodiments so that those skilled in the art may better understand the detailed description. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

1. A system for interfacing cellular matter with a machine, comprising:
a transponder implantable in cellular matter and configured to detect a first signal in the matter;
a receiver external to the cellular matter and configured to receive a second signal transmitted in response to the transponder detection of the first signal; and
a transmitter configured to transmit a third signal to a machine in response to the receiver receipt of the second signal.

2. The system of claim 1 wherein the transponder is remotely powered.

3. The system of claim 1 wherein the transponder is remotely powered using radio frequency (RF) propagation.

4. The system of claim 1 further comprising a processor in electrical communication with the receiver and the transmitter.

5. The system of claim 1 wherein at least two of the first, second and third signals are substantially similar.

6. The system of claim 1 wherein the transponder is a plurality of transponders.

7. The system of claim 1 wherein the receiver is a plurality of receivers.

8. The system of claim 1 wherein the receiver is configured to detect a unique source corresponding to the detected first signal.

9. The system of claim 1 further comprising the machine.

10. The system of claim 1 wherein the machine is a computer.

11. The system of claim 1 wherein the machine is a motor controller.

12. The system of claim 1 wherein the machine is an actuator.

13. The system of claim 1 wherein the machine is a music synthesizer.

14. The system of claim 1 wherein the machine is a speech synthesizer.

15. The system of claim 1 wherein the machine is a navigational control system.

16. The system of claim 1 wherein the machine is a graphical display unit.

17. The system of claim 1 wherein the cellular matter is living human tissue.

18. The system of claim 17 wherein the tissue is brain tissue.

19. The system of claim 1 wherein the cellular matter is human blood.

20. The system of claim 1 further comprising a helmet housing at least one of the receiver and the transmitter.

21. The system of claim 1 further comprising a hand-held device housing at least one of the receiver and the transmitter.

22. The system of claim 1 further comprising support structure housing at least one of the receiver and the transmitter and having a open scanning region through which the cellular matter is transportable.

23. The system of claim 3 further comprising a coil external to the cellular matter and employed to remotely power the transponder.

24. The system of claim 23 wherein the coil is used to remotely power the transponder and the receiver receives the second signal simultaneously.

25. The system of claim 1 wherein the transponder has a volume less than or equal to 1 cubic millimeter.

26. The system of claim 1 wherein the transponder has a maximum lateral dimension that is not greater than about 1000 microns.

27. The system of claim 1 wherein the transponder has a maximum lateral dimension that is not greater than about 200 microns.

28. The system of claim 1 further comprising an enclosure housing at least one of the receiver and the transmitter.

29. The system of claim 28 wherein the receiver includes a reader coil and the transponder includes a transponder coil and the enclosure is positioned relative to the cellular matter such that the reader coil has a predetermined orientation relative to the transponder coil.

30. The system of claim 29 wherein the predetermined orientation of the reader coil comprises an offset spacing from the transponder coil of about three centimeters or less.

31. The system of claim 29 wherein the transponder coil and the reader coil are magnetically coupled.

32. The system of claim 31 wherein the second signal is transmitted to the receiver via the magnetic coupling of the transponder coil and the reader coil.

33. The system of claim 32 wherein the transponder is remotely powered by radio frequency propagation via the magnetic coupling of the reader coil and the transponder coil.

34. The system of claim 32 further comprising a power coil housed in the enclosure and magnetically coupled with the transponder coil, wherein the transponder is remotely powered by radio frequency propagation via the magnetic coupling of the power coil and the transponder coil.

35. The system of claim 1 wherein the first signal is a biological signal.

36. A system for interfacing cellular tissue with a machine, comprising:
means implantable in cellular matter for detecting a first signal in the cellular matter;
means for receiving a second signal transmitted in response to the detection of the first signal; and
means for transmitting a third signal to a machine in response to the receipt of the second signal.

37. A transponder, comprising:
a remote-activated power source;
a sensor powered by the power source and configured to detect a first signal propagating in cellular matter; and
a transmitter configured to transmit a second signal in response to detection of the first signal by the sensor;
wherein the transponder is implantable in the cellular matter.

38. The transponder of claim 37 wherein the transponder has a maximum lateral dimension of about 200 μm.

39. The transponder of claim 37 further comprising an enclosure housing the power source, the sensor and the transmitter and having an outer surface coated with a bioprotectant.

40. The transponder of claim 37 wherein the power source is activated by radio-frequency propagation.

41. The transponder of claim 37 further comprising an antenna in electrical communication with the sensor and couplable to a signal detector external to the transponder.

42. The transponder of claim 37 wherein the first signal is a biological signal.

43. A method for controlling a machine in response to signals propagating in cellular matter, comprising:
detecting a first signal in cellular matter;
transmitting a second signal in response to the detection of the first signal; and
transmitting a third signal to a machine in response to the receipt of the second signal.

44. The method of claim 43 further comprising operating the machine to perform a function having a result associated with the detected first signal.

45. The method of claim 43 further comprising implanting a transponder in the cellular matter, the transponder configured to perform the detecting the first signal and the transmitting the second signal.

46. The method of claim 43 further comprising sensing the result of the function of the machine operation.

47. The method of claim 46 further comprising detecting a fourth signal in the cellular matter in response to the sensing the result of the function performed by the machine.

48. The method of claim 43 wherein the cellular matter is human brain tissue.

49. The method of claim 48 wherein the machine is a motor controller in electrical communication with a motor.

50. The method of claim 49 wherein the motor is connected to a wheelchair wherein the result of the function of the motor controller is movement of the wheelchair.

51. The method of claim 43 wherein the cellular matter is a part of an animal.

52. The method of claim 43 further comprising locating a transponder from which the second signal originates.

53. The method of claim 52 wherein locating the transponder includes identifying one of a plurality of transponders.

54. An interface between cellular matter and a machine, comprising:
a housing;
a receiver coupled to the housing and configured to receive a first signal from a transponder implanted in cellular matter, wherein the receiver is external to the cellular matter; and
a transmitter coupled to the housing and configured to transmit a second signal to a machine in response to the receiver receipt of the first signal.

55. The interface of claim 54 wherein the housing is a helmet.

56. The interface of claim 54 wherein the housing is a hand-held device.

57. The interface of claim 54 wherein the transmitter wirelessly transmits the second signal to the machine.
